(19) United States
(12) Patent Application Publication
(10) Pub. No.: US 2020/0367805 A1
(43) Pub. Date: Nov. 26, 2020

(54) METHODS AND APPARATUSES FOR DETECTING CANCEROUS TISSUE

(71) Applicant: The Government of the United States, as represented by the Secretary of the Navy, Washington, DC (US)

(72) Inventors: Kenneth Ewing, Edgewood, MD (US); Kevin Major, Alexandria, VA (US); Jas Sanghera, Ashburn, VA (US)

(21) Appl. No.: 16/989,792

(22) Filed: Aug. 10, 2020

Related U.S. Application Data
(63) Continuation-in-part of application No. PCT/US19/17166, filed on Feb. 8, 2019.
(60) Provisional application No. 62/627,989, filed on Feb. 8, 2018.
(60) Provisional application No. 62/925,006, filed on Oct. 23, 2019.

(51) Int. Cl. A61B 5/00

(52) U.S. Cl. CPC A61B 5/444; A61B 2562/0238; A61B 5/0075

(57) ABSTRACT

Methods and apparatuses for detecting cancerous tissue are provided. A target area comprising suspected tissue is illuminated with infrared light. Reflected light from the target area is received and filtered by a plurality of infrared optical filters with overlapping pass-bands. The filtered reflected light from each of the plurality of optical infrared filters is directed onto at least one optical sensor, and data generated by the at least one optical sensor and corresponding to the filtered reflected light from each of the plurality of optical infrared filters is provided to a controller. The controller calculates a vector corresponding to the target area from the data, and compares that vector to a plurality of vectors respectively corresponding to different types of cancerous tissue. The controller determines based on a result of the comparison whether the target area includes at least one type of cancerous tissue.

### BACKGROUND

### Field of the Invention

The present application relates generally to apparatuses and methods for detecting cancerous tissue.

### Description of Related Art

Skin cancer is responsible for the deaths of over 12,000 Americans each year. Melanoma is a particularly deadly type of skin cancer as it is responsible for 75% of all deaths, even though it only accounts for 4% of skin cancer cases. Skin cancer is currently detected by a two-step process. First, a patient is visually inspected by a care provider. The provider uses the “ABCDE” (Asymmetry, Border, Core, Diameter, and Evolution) test to identify potentially cancerous growths. The ABCDE calls for the provider to scan the patient's body and make an educated guess at whether suspicious tissue is cancerous, pre-cancerous, or normal healthy skin. If the provider believes the tissue is cancerous, then a biopsy is performed to provide a definitive determination. If the provider believes the tissue is normal healthy skin, no biopsy is performed. The ABCDE test, however, is problematic. If the provider incorrectly identifies suspicious tissue as normal healthy skin, when in fact it is cancerous, then the cancer may spread throughout the rest of the body and ultimately lead the patient's death. Because of the severe consequences of misidentifying cancerous tissue as healthy tissue, providers err on the side of caution and perform a biopsy when in doubt. It is therefore not surprising that the ABCDE test exhibits a false positive rate of about 35-40%. What this means is that each year thousands of unnecessary biopsies are performed.

While others have considered using infrared light to detect cancerous growths, those systems require elaborate and expensive equipment that requires significant training to operate. As such, they are seldom if ever used in a clinical setting. It would therefore be desirable to have methods and apparatuses for non-invasive detection of skin cancer that are simple to use and cost effective.

### SUMMARY OF THE INVENTION

One or more the above limitations may be diminished by structures and methods described herein.

In one embodiment, a method for detecting cancerous tissue are provided. A target area comprising suspected tissue is illuminated with infrared light. Reflected light from the target area is received and filtered by a plurality of infrared optical filters with overlapping pass-bands. The filtered reflected light from each of the plurality of optical infrared filters is directed onto at least one optical sensor, and data generated by the at least one optical sensor and corresponding to the filtered reflected light from each of the plurality of optical infrared filters is provided to a controller. The controller calculates a vector corresponding to the target area from the data, and compares that vector to a plurality of vectors respectively corresponding to different types of cancerous tissue. The controller determines based on a result of the comparison whether the target area includes at least one type of cancerous tissue.

In another embodiment, an apparatus for detecting cancerous tissue is provided. The apparatus includes an infrared light source, a plurality of mid-wavelength infrared optical filters, at least one optical sensor, and a controller. The infrared light source is configured to direct infrared light onto a target area comprising suspected tissue. The plurality of mid-wavelength infrared optical filters are configured to receive reflected light from the target area and have overlapping pass-bands. The least optical sensor is configured to receive filtered reflected light from each of the plurality of optical infrared filters. The controller is configured to: receive data generated by the at least one optical sensor and corresponding to the filtered reflected light from each of the plurality of optical infrared filters, calculate a vector corresponding to the target area from the data, compare the vector corresponding to the target area with a plurality of vectors respectively corresponding to different types of cancerous tissue, and determine based on a result of the comparison whether the target area includes at least one type of cancerous tissue.

### BRIEF DESCRIPTION OF THE DRAWINGS

The teachings claimed and/or described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1A is a schematic view of an apparatus for detecting cancerous tissue according to one embodiment.

FIG. 1B is a schematic view of an apparatus for detecting cancerous tissue according to another embodiment.

FIG. 1C is a schematic view of an apparatus for detecting cancerous tissue according to yet another embodiment.

FIG. 2A illustrates reflected light passing through a plurality of optical filters onto an optical sensor according to one embodiment.

FIG. 2B is a plan view of the plurality of optical filters overlaid on the optical sensor according to one embodiment.

FIG. 2C is a schematic view of a rotatable member holding a plurality optical filters according to one embodiment.

FIG. 2D illustrates reflected light passing through a plurality of optical filters onto a plurality of optical sensors according to one embodiment.

FIG. 2E is a plan view of a plurality of optical filters overlaid on a plurality of optical sensors.

FIG. 3 illustrates reflected light passing through a plurality of optical filters that are separated by light isolating structures, according to one embodiment.

FIG. 4 is a flowchart illustrating steps in analyzing a target area comprising suspicious tissue.

FIG. 5 is a graph illustrating the pass-band regions of a plurality of optical filters, according to one embodiment.

FIG. 6 is a graph illustrating the pass-band regions of a plurality of optical filters and a spectrum generated by scanning normal healthy skin.

FIG. 7 is a graph illustrating the pass-band regions of a plurality of optical filters and experimentally recorded spectrum from normal healthy skin.

FIG. 8 is a graph illustrating the pass-band regions of a plurality of optical filters and experimentally recorded spectrum from nodular melanoma.

FIG. 9 is a graph illustrating the pass-band regions of a plurality of optical filters and experimentally recorded spectrum from superficial spreading melanoma.

FIG. 10 is a graph illustrating the pass-band regions of a plurality of optical filters and experimentally recorded spectrum from melanoma metastasis.

FIG. 11 is a three-dimensional view of vectors corresponding to normal healthy skin and nodular melanoma.

FIG. 12 is a three-dimensional view of vectors corresponding to normal healthy skin and superficial spreading melanoma.

FIG. 13 is a three-dimensional view of vectors corresponding to normal healthy skin and melanoma metastasis.

FIG. 14 is a chart illustrating another method of determining whether a scanned region is healthy skin or cancerous.

Different ones of the Figures may have at least some reference numerals that are the same in order to identify the same components, although a detailed description of each such component may not be provided below with respect to each Figure.

### DETAILED DESCRIPTION OF THE PREFERRED EMBODIMENTS

In accordance with example aspects described herein are methods and apparatuses for non-invasive skin cancer detection.

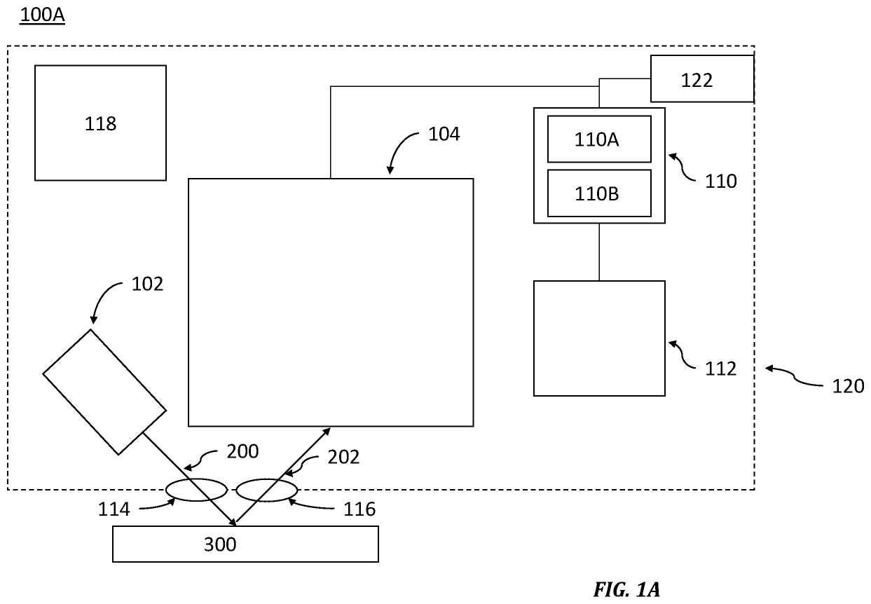

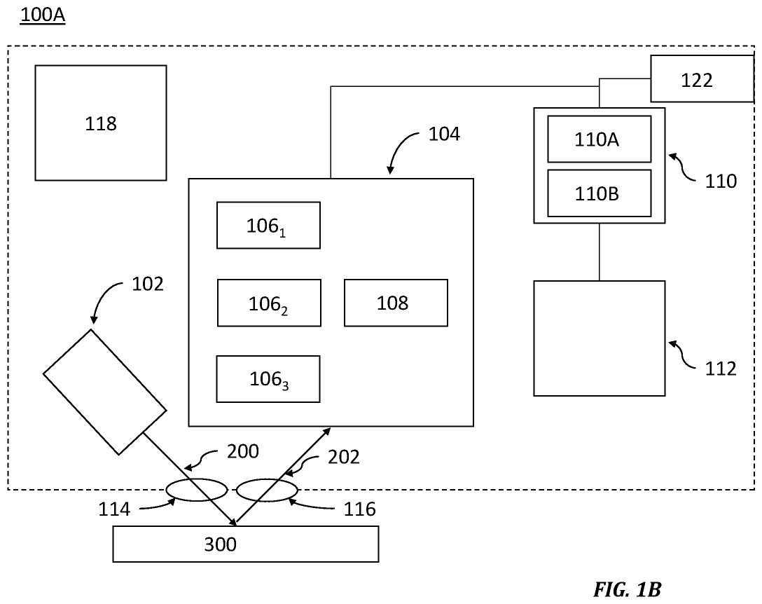

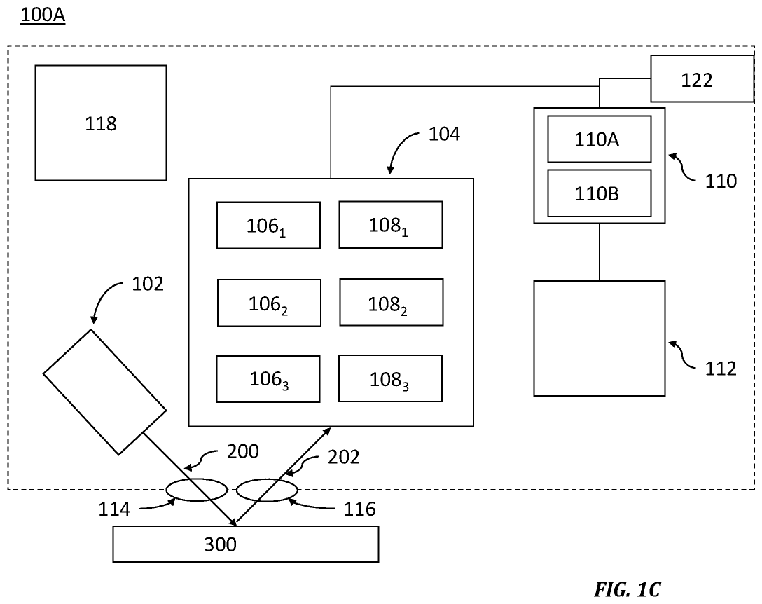

FIG. 1A is a schematic diagram of a cancer detection system 100A for non-invasively detecting skin cancer according to one embodiment. The cancer detection system 100A includes a source 102, a detection section 104, a processor 110, a notification device 112, a first aperture 114, a second aperture 116, a power supply 118, a housing 120, and an I/O connection 122. Each of these components may be easily housed within a housing 120 resulting a portable that can be hand-carried. Each of these components will be discussed in detail below.

In one embodiment, source 102 is an infrared light source transmitting long wavelength infrared light (LWIR) or medium wavelength infrared light (MWIR). LWIR may be considered wavelengths 8-15 μm, and MWIR may be considered 2-8 μm. In one embodiment, source 102 transmits the infrared light through an optical fiber (not shown), such as a chalcogenide optical fiber, that extends through the first aperture 114. The optical fiber may be enclosed by in a flexible cladding that allows the fiber to be repositioned with minor effort, but holds its position once the operator (e.g., the provider) places it in the desired position. This allows the optical fiber to be placed proximate to a target area 300 that is being evaluated. In another embodiment, light 200 from source 102 is transmitted through a lens (not shown) that focuses the light 200 on to the target area 300. Regardless of the method of delivery, infrared light 200 is directed onto the target area 300, and reflected light 202 from the target area 300 returns to the detection system 100A via aperture 116. One of the principles behind the operation of system 100A is that different tissues in the target area 300 will absorb infrared light differently. Healthy skin, for example, may absorb certain wavelengths of infrared light that are not equally absorbed by cancer cells. The recorded spectrum of infrared light reflected from healthy skin will therefore be different from the recorded spectrum of infrared light reflected cancerous cells. However, the recorded spectra will be quite similar and thus difficult to distinguish from each other without further analysis. Thus, in system 100A, the reflected light 202 is provided to a detection section 104 constructed to aid in the discrimination analysis.

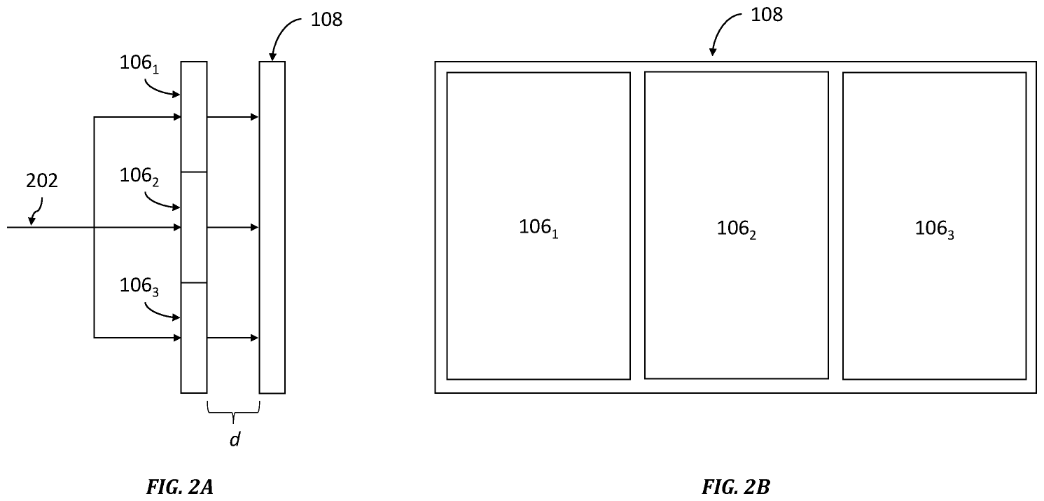

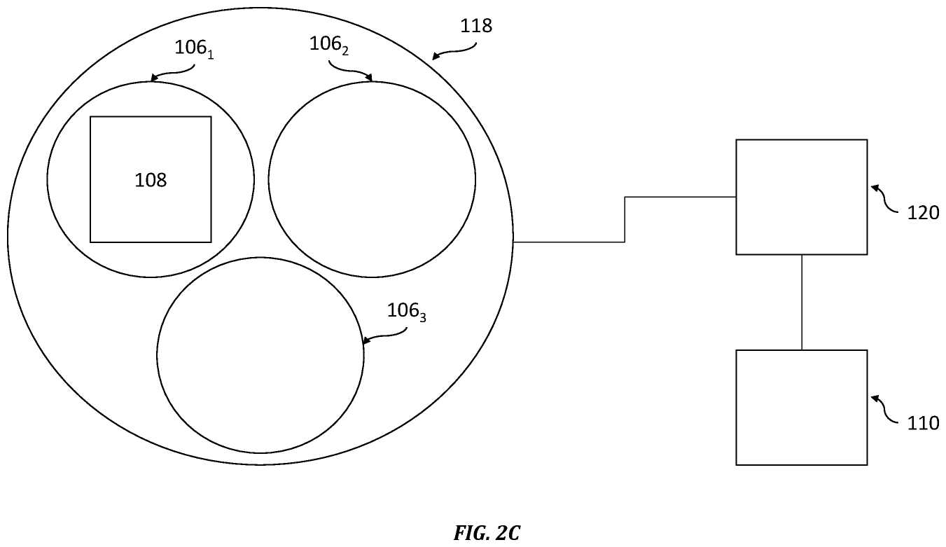

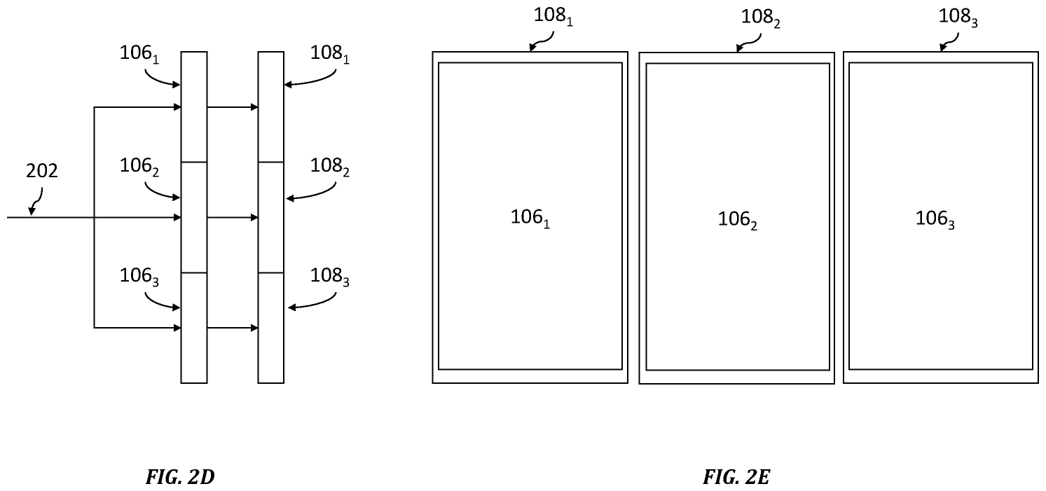

Detection section 104 functions to convert the reflected light 202 into data that is analyzed by processor 110. Detection section 104 may be implemented in different embodiments. However, each embodiment includes a plurality of optical filters, generically 106ᵢ, and at least one optical sensor 108. FIGS. 1B-2D illustrate different embodiments of the detection section 104 which are discussed below.

FIGS. 1B, 2A, and 2B illustrate one embodiment of the detection section 104 that includes a plurality of optical filters 106₁ . . . 106₃ and one optical sensor 108. As shown in FIG. 2A, reflected light 202 from the target area 300 that is received through aperture 116 is split into a number of beams corresponding to the number of optical filters 106ᵢ. As one of ordinary skill will appreciate, this may be accomplished by the use of well-known optical components including beamsplitters and mirrors. Each optical filter 106₁ . . . 106₃ is constructed to allow at least partial transmission of a certain wavelength range of the reflected light 202, as discussed in detail below. In the embodiment shown in FIGS. 1B, 2A, and 2B, filtered reflected light 202 from optical filters 106₁ . . . 106₃ is incident on a single optical sensor 108. Optical sensor 108 is constructed to sense infrared light and may be a PbSe image sensor, Si or VOₓ microbolometer or infrared focal plane array (FPA), HgCdTe (MCT), Deuterated triglycine sulfate detector (DTGS), InSb, InAs, infrared photodiode, or other sensor operating in the MWIR or LWIR.

FIGS. 2A and 2B illustrate one embodiment of filters 106₁ . . . 106₃ and optical sensor 108. In FIGS. 2A and 2B, optical sensor 108 is positioned relative to filters 106₁ . . . 106₃ so that filtered light from each of the optical filters 106₁ . . . 106₃ is incident on a different section of the optical sensor 108. This may be accomplished by selecting an optical sensor 108 who area is greater than sum of the areas of optical filters 106₁ . . . 106₃, and placing optical sensor 108 a distance d from the optical filters 106₁ . . . 106₃ such that filtered light from each filter 106₁ . . . 106₃ falls entirely within its corresponding section of optical sensor 108, as illustrated in FIG. 2B.

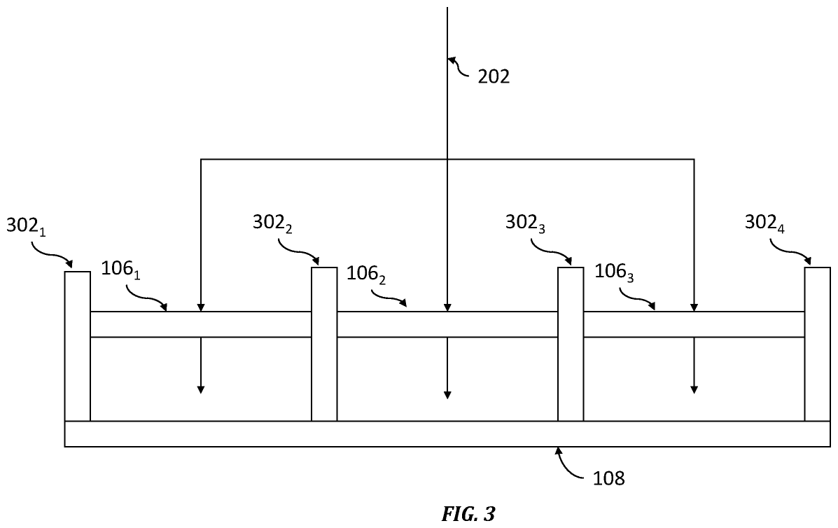

Filters 106₁ . . . 106₃ may also be placed at a distance greater than d from the optical sensor 108, if light isolation pillars 302₁ . . . 302₄, made from materials that absorb infrared light are provided to confine the filtered light from filters 106₁ . . . 106₃ to their corresponding areas of sensor 108, as shown in FIG. 3. This arrangement prevents filtered reflected light 202 from one optical filter 106₁ . . . 106₃ from being incident on a portion of the optical sensor 108 designated for receiving light from another of the optical filters 106₁ . . . 106₃. For example, isolation pillars 302₁ and 302₂ prevent filtered light from optical filter 106₁ from escaping and being incident on a portion of the optical sensor 108 designated for receiving light from optical filters 1062 or 106₃. One of the advantages of this configuration is that the voltages generated on sensor 108 in response to the light form filters 106₁ . . . 106₃ may be readout simultaneously under the control of controller 110.

In another embodiment, the detection section 104 may still include plurality of optical filters 106₁ . . . 106₃ and one optical sensor 108, but the filters may be mounted on a rotatable holder 118 that is constructed to be rotated between three different positions by a drive motor 120, operating under the control of controller 110, as shown in FIG. 2C. Each position places a different optical filter 106₁ . . . 106₃ in the path of the reflected light 202 upstream from sensor 108. Thus, under the control of controller 110, the reflected light 202 passes through one of the filters 106₁ . . . 106₃ and is incident on the sensor 108 for a predetermined period called the capture time. After the capture time, the accumulated voltages on sensor 108 are readout under the control of controller 110 and converted to a digital signal for analysis by controller 110, as described below. Controller 110 may then instruct drive motor 120 to rotate the holder 118 to another position such that a different filter 106₁ . . . 106₃ is placed in the path of the reflected light 202, and the above process is repeated. Controller 110 then instructs drive motor 120 to rotate the holder 118 to the last position such that the last remaining filter 106₁ . . . 106₃ is in the path of reflected light 202, and the above process is repeated for a final time. Since the composition of the target area 300 is static for the period over which the holder 118 is rotated between the three positions and the data is collected, the spectrum of the reflected light 202 will remain constant over that period as well. One of the advantages of this configuration is that the beam of reflected light 202 need not be subdivided into separate beams for each optical filter 106₁ . . . 106₃. Consequently, the number of optical components required for system 100A is reduced leading to a reduction in cost and size.

In another embodiment, the detection section 104 may include a plurality of optical filters 106₁ . . . 106₃ respectively corresponding to a plurality of optical sensors 108₁ . . . 108₃, as illustrated in FIGS. 1C, 2D, and 2E. In this embodiment, the isolation pillars 302 shown in FIG. 3 are unnecessary as light emanating from each optical filter 106₁ . . . 106₃ is incident on its own optical sensor 108₁ . . . 108₃. Like in the embodiments described above, voltages produced in sensors 108₁ . . . 108₃ are readout under the control of controller 110 at the end of the accumulation period. The voltages are then converted into a digital signal for analysis by controller 110 as described below.

Regardless of the implementation of detection section 104, data in the form a digital signal is analyzed by controller 110. Controller 110 includes a processor 110A which may be a central processing unit (CPU), a microprocessor, or a microcontroller. Controller 110 also includes memory 110B. Memory 110B stores a control program that, when executed, causes processor 110A to perform the analysis described below. As discussed in more detail below, memory 110B also stores a plurality of vectors corresponding to different types of cancer and may also store at least one vector corresponding to normal healthy skin. Memory 110B also includes storage space for storing the results of the analysis and temporary data generated in the course of the analysis.

Finally, notification device 112 is constructed provide a notification to the user of the result of the analysis. In a preferred embodiment, notification device 112 is display constructed to provide the operator with the result of the analysis. The display may be a touch screen display capable of receiving inputs, like a start instruction from the operator. If processor 110A is able to determine that the target area 300 is a specific type of cancer or healthy skin, then the same information may be displayed on the display. If system 100A is unable to match the target area 300 to one of the types of cancer or healthy skin, then the notification device may display a message indicating that no match was found. Finally, notification device 112 may also be configured to provide support information to the operator. For example, system 100A may be initially calibrated by scanning a known reference material (e.g., gold foil) whose absorption properties in the infrared are known. Using the analysis described below, if processor 110A is able to match a vector corresponding to target area 300 with a vector for roughened gold, stored in memory 110B, to a predetermined degree of accuracy, then processor 110A may instruct the notification device display a message that system 100A is successfully calibrated. If, however, processor 110A is unable to match the reference material in target area 300 to the vector for the same stored in memory 110B, then processor 110A may instructed the notification device 112 to display a message indicating that the calibration for system 100A has failed, and instruct the operator to contact a service provider. This calibration helps to ensure that the system 100A has the requisite accuracy to discriminate between cancerous tissue and normal tissue. Having described the various components of system 100A, a method of using the same to discern the nature of a target area 300 will now be described.

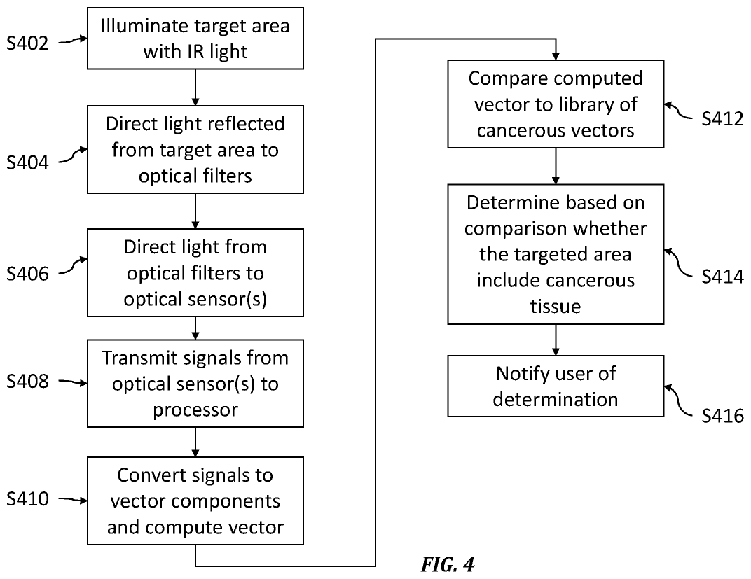

FIG. 4 illustrates a process of using the detection system 100A to discern the nature of a target area 300. A start instruction is received from the operator. The start instruction may be received through notification device 112 (e.g., notification device 112 may be a touchscreen display). System 100A may also include physical buttons (not shown) on the periphery of housing 120 to allow for operation of system 100A.

Detection system 100A may also include an I/O connection 122 that allows for communication between controller 110 and a connected device. The I/O connection 122 may be, for example, a serial connection or a USB connection for receiving the start instruction, and other commands, from another computer. Data and instructions may be sent to and from the controller 110 via the I/O connection 122. For example, data from sensor(s) 108 may be transmitted to the connected device through I/O connection 122. In addition, updates to the control program or the database of vectors corresponding to different types of cancer and healthy skin that are stored in memory 110B may be updated or modified through the I/O connection 122.

Upon receipt of the start instruction from either the notification device 112, the physical buttons on housing 120, or the I/O connection 122, processor 110A executes the control program stored in memory 110B to begin the sequence of steps illustrated in FIG. 4. Under the control of controller 110, source 102 illuminates the target area 300 for a predetermined period of time, in S402. The time in which the target area 300 is illuminated with infrared light is set in the control program, but may be modified through the notification device 112, the physical buttons on the periphery of the housing 120, or the I/O connection 122. The time in which the target area is illuminated is sufficient to allow for generating the necessary voltages in the one or more sensors 108ᵢ such that data output from the one or more sensors 108ᵢ has a high signal/noise ratio.

Reflected light 202 is received from the target area 300 through aperture 116 in the detection system 100A and directed to the optical filters 106₁ . . . 106₃ in S404. As discussed above, depending on the implementation of the optical filters 106₁ . . . 106₃ and the optical sensor(s) 108ᵢ, the optical components for directing the reflected light 202 to the filters 106₁ . . . 106₃ may vary. For example, in the case of the embodiment shown in FIG. 2C, a single mirror may be used to direct the reflected light 202 received through aperture 116 on a path towards the rotatable holder 118 and the optical sensor 108. However, in the embodiments shown in FIGS. 2A and 2D, at least two beamsplitters will be required to split the reflected light 202 into three separate beams. Having described how the reflected light from the target area 300 is received and directed to the optical filters 106₁ . . . 106₃, the function of the filters 106₁ . . . 106₃ themselves will now be discussed.

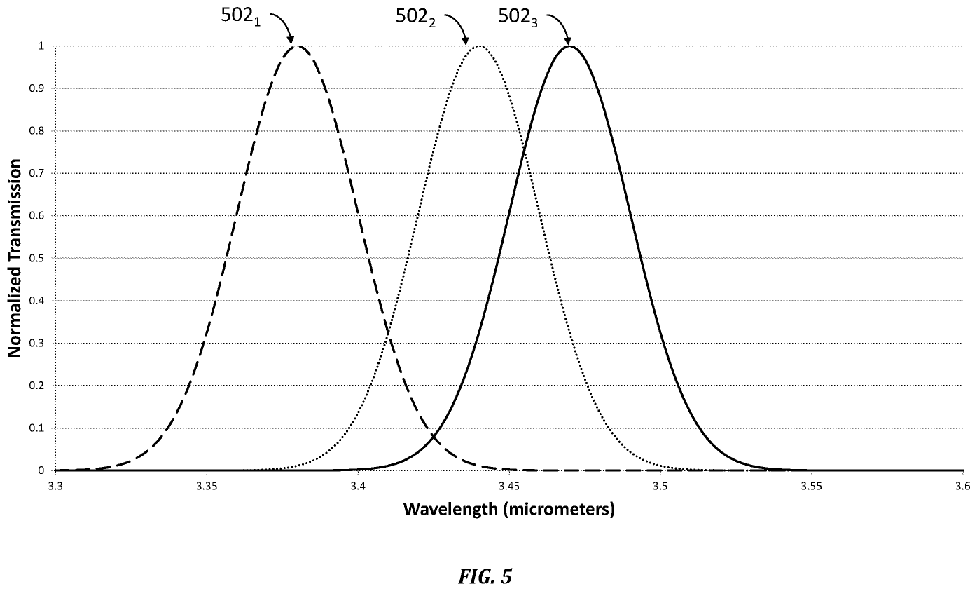

Each optical filter 106₁ . . . 106₃ is designed to allow a certain wavelength range, called the pass-band, to pass through the filter while blocking the transmission of light that falls outside of the pass-band. However, each wavelength of light within the pass-band need not, and preferably are not, transmitted equally, FIG. 5 is illustrative. FIG. 5 shows normalized transmission profiles 502₁ . . . 502₃ respectively corresponding to filters 106₁ . . . 106₃. Profiles 502₁ . . . 502₃ are centered on wavelengths 3.38 μm, 3.44 μm, and 3.47 μm respectively. These filter profiles 502₁ . . . 502₃ have been demonstrated (as discussed below) to be capable of discriminating between cancerous and non-cancerous tissue. However, the location of these filter profiles 502₁ . . . 502₃ need not be fixed at these centerpoints and may be shifted slightly within the MWIR. The higher the transmission value (i.e., the closer to 1) is for a particular wavelength, the more the filter will allow that particular wavelength to pass. Thus, transmission is highest for the centerpoint wavelengths indicated above, and those wavelengths will pass through the respective filters 106₁ . . . 106₃ without any attenuation. However, as the wavelength of reflected light 202 moves away from the centerpoint wavelengths, the reflected light 202 becomes more attenuated until, at the ends of the pass-band, transmission is blocked.

As is evident from FIG. 5, filter profiles 502₁ . . . 502₃ overlap with one another. In fact, these overlapping filter profiles 502₁ . . . 502₃ mimic the response of the human eye to visible light. In the human eye, color discrimination is accomplished by using three different pigments contained in the cone cells of the retina. These pigments exhibit broad absorption bands and significant spectral overlap. Each of the pigments has a varying response to different wavelengths of light. When light enters the eye, it interacts with these pigments based on the spectral wavelength overlap of the incoming light and each pigment. The combination of the output for the three different cone cells enables the identification and discrimination of different colors without the use of a spectrometer. System 100A operates in a similar way. The filter profiles 502₁ . . . 502₃ are analogous to the different pigments in the human eye. In the same way each pigment reacts differently to different wavelengths of light, so to do the filter profiles 502₁ . . . 502₃ transmit reflected light differently depending on wavelength. The filter responses, that is the output of the filters, will therefore vary as the spectrum of the reflected light 202 changes. For example, if the target area 300 consists a first material that strongly absorbs infrared light, one would expect to see one set of filter responses. However, if the target area 300 consists of a second material that weakly absorbers infrared light, one would expect to see a different set of filter responses. Thus, by analyzing the output of the overlapping filters 106₁ . . . 106₃, system 100A is able to discriminate between different materials in the target area 300, as explained in greater detail below.

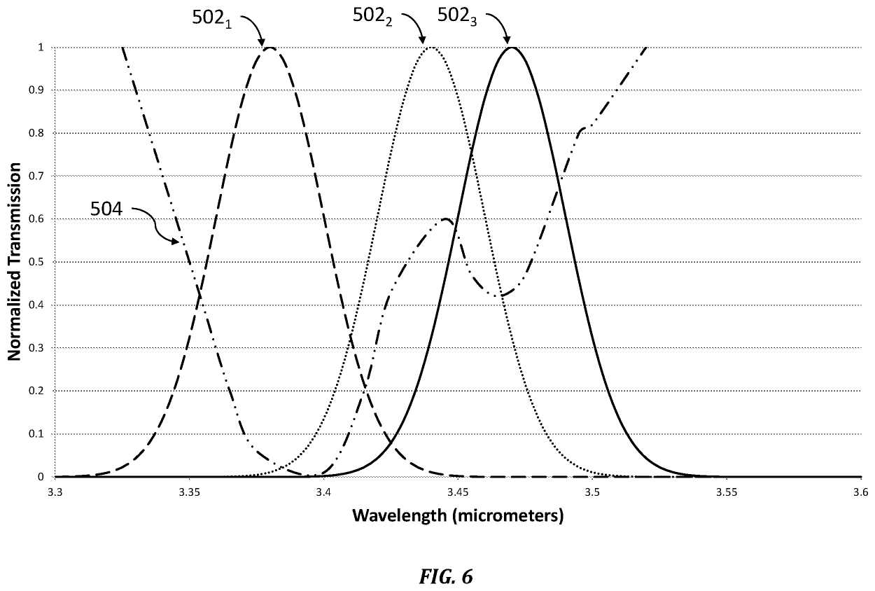

FIG. 6 shows the filter profiles from FIG. 5 along with normalized MWIR spectra 504 recorded from a target area 300 comprising healthy skin 504, prior to passing through any filters. As shown in FIG. 6, certain wavelengths show transmission values below 1 indicating that those wavelengths were absorbed by the material (tissue) in target area 300. With this information, a computer may calculate a vector coordinate set {v₁, v₂, v₃}, also called vector components, for spectrum 504 in a space defined by the normalized vector components for each filter 106₁ . . . 106₃. This is done by integrating the product of the infrared transmission spectrum 504 by the filter profiles 502₁ . . . 502₃ for each filter 106₁ . . . 106₃, as shown in Equation 1 below:

∫_{λ}_{₂}^{λ}^{₁}F₁₀₆_{ᵢ}(λ)S(λ)dλ

In Equation 1, F₁₀₆_{ᵢ} represents each filter profile 502₁ . . . 502₃, SQL) is spectrum 504, λ₁ is one end of spectrum 504 (e.g., 3.325 in FIG. 6), and λ₂ is the other end of the spectrum 504 (e.g., 3.52 in FIG. 6). However, performance of these calculations is made unnecessary by system 100A because the voltages readout from the one or more optical sensors 108ᵢ directly correspond to the three vector components. Thus, the voltages readout from sensor 108, or a portion thereof, corresponding to filter 106₁ directly corresponds to the vector component for filter 106₁. Processor 110A may therefore generate the vector components directly from the data recorded by the optical sensor(s) 108₁ without the need for performing the integral in Equation 1. This is done by processor 110A calculating the difference (v_{diff-106-i}) between the recorded voltage for each filter (v₁₀₆₋ᵢ) and a background voltage for each filter (v_{background}). The difference voltages for each filter, v_{diff-106-1}, v_{diff-106-2}, and v_{diff-106-3} are then normalized, by processor 110A, such that the sum of their square roots is 1. Thus, the corresponding vector coordinate set (v₁₀₆₋₁, v₁₀₆₋₂, and v₁₀₆₋₃) will have a unit length of 1, as shown in Equations 2-4 below.

$${v}_{106 - 1} = \frac{{V}_{diff - 106 - 1}}{\sqrt{( {V}_{diff - 106 - 1}^{2} + {V}_{diff - 106 - 2}^{2} + {V}_{diff - 106 - 3}^{2} )}}$$

$${v}_{106 - 2} = \frac{{V}_{diff - 106 - 2}}{\sqrt{( {V}_{diff - 106 - 1}^{2} + {V}_{diff - 106 - 2}^{2} + {V}_{diff - 106 - 3}^{2} )}}$$

$${v}_{106 - 3} = \frac{{V}_{diff - 106 - 3}}{\sqrt{( {V}_{diff - 106 - 1}^{2} + {V}_{diff - 106 - 2}^{2} + {V}_{diff - 106 - 3}^{2} )}}$$

There are two advantages of this approach. First, as mentioned above, there is no need for a spectrometer to record the spectrum of the reflected light 202, as S(λ) is not required to generate the vector components. This reduces the cost, size and complexity of system 100A. Second, since it is not necessary to calculate Equation 1 to generate the vector components, the computational requirements for processor 110A are reduced. Again, this allows for lower-speed and cheaper processors to be used without any loss of system performance. Moreover, since Equation 1 does not have to be calculated, system 100A is able to provide a discrimination result faster than a system that uses a spectrometer, enhancing the speed of system 100A as a whole. The function of filters 106₁ . . . 106₃ will now be further explained in the context of experimental data below.

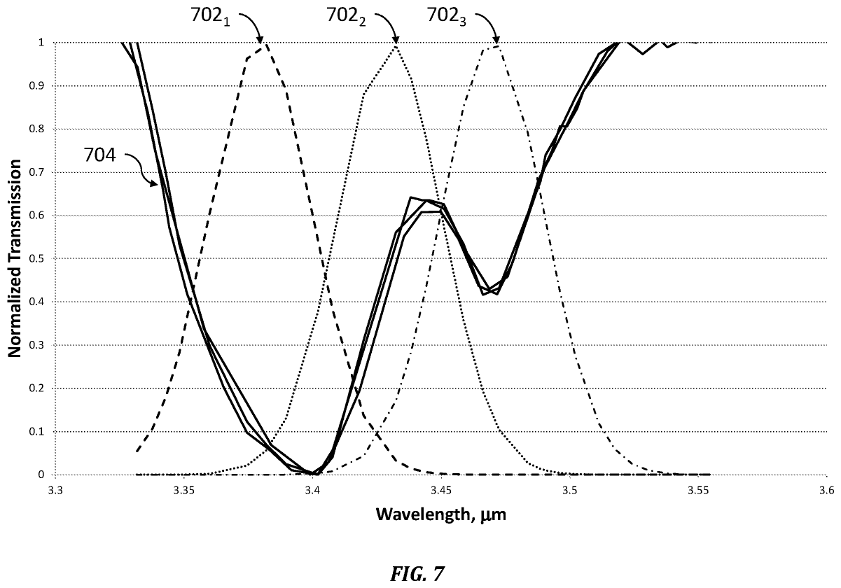

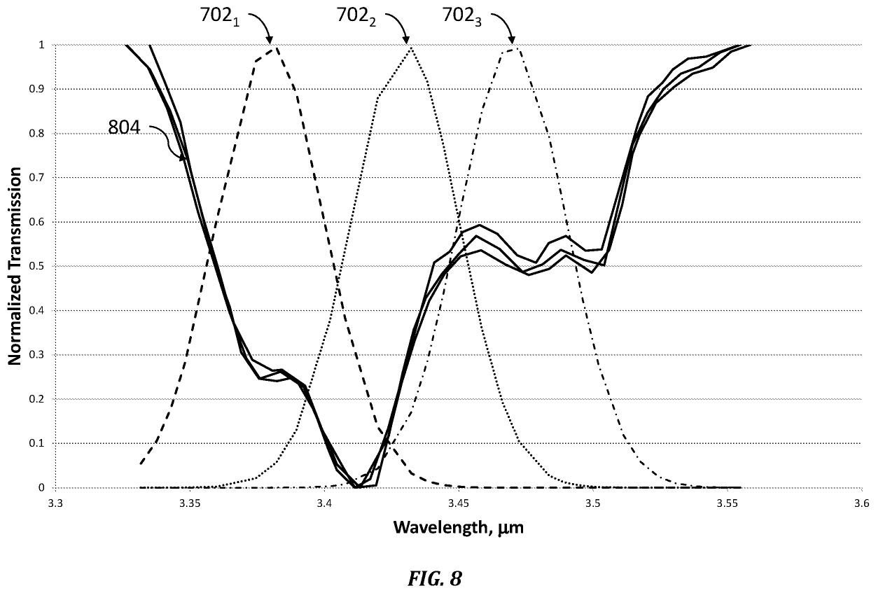

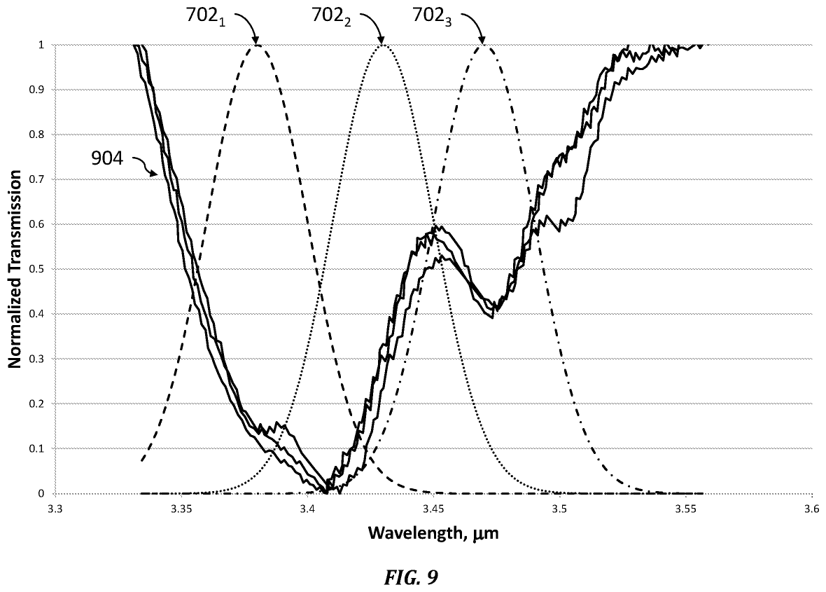

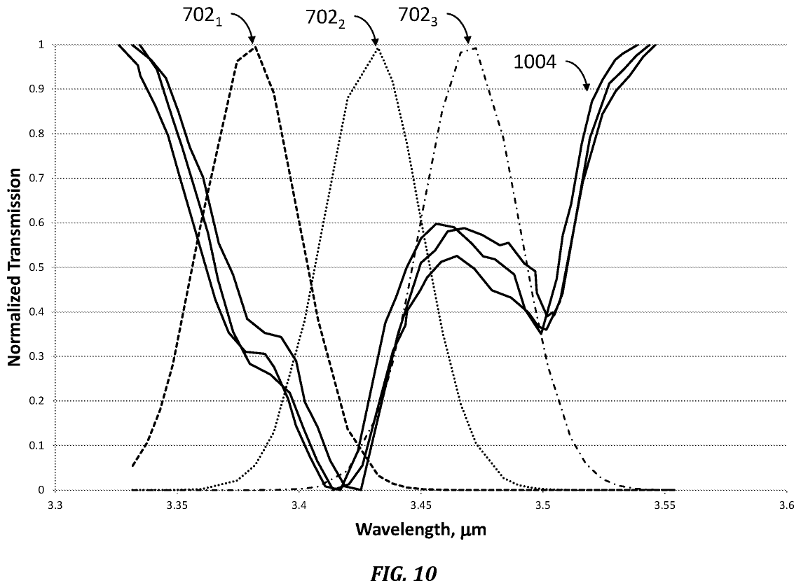

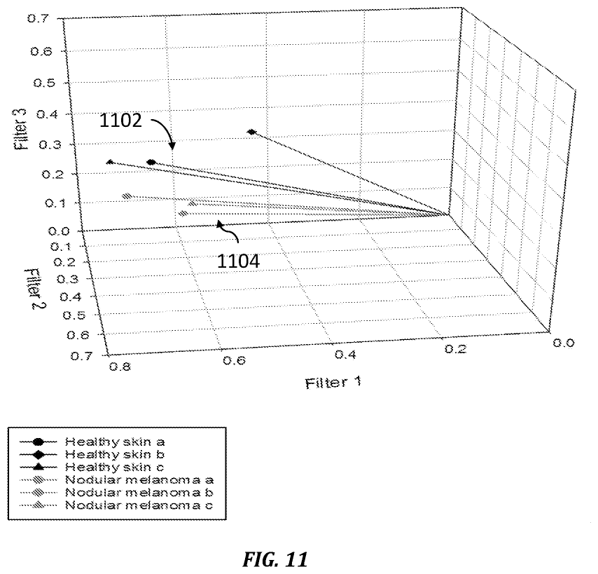

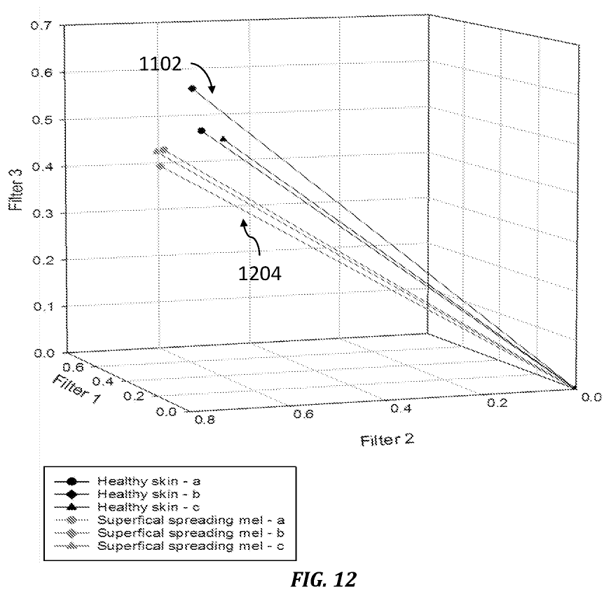

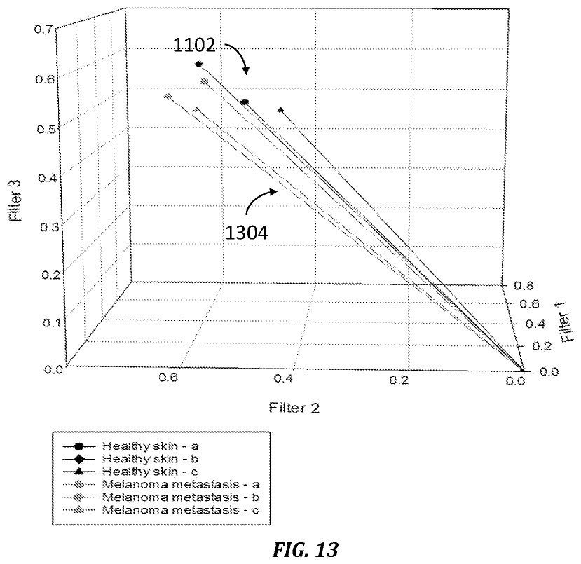

FIGS. 7-10 show experimental data obtained from scanning normal healthy skin (FIG. 7), nodular melanoma (FIG. 8), superficial spreading melanoma (FIG. 9), and melanoma metastasis (FIG. 10). In each of FIGS. 7-10, three different targets areas (A, B, and C) 300 were scanned, resulting in three target spectra groups 704, 804, 904, and 1004 in each figure, respectively. As noted above, system 100A does not have to record the spectrum from the target area 300, and these spectras are provided merely to illustrate the different spectrums for different types of cancer and normal skin. Also shown in FIGS. 7-10 are the three optical filters profiles 701₁ . . . 701₃ respectively corresponding to filters 106₁ . . . 106₃, that were used to analyze the experimental data. Here, the filter profiles 701₁ . . . 701₃ are centered and shaped the same as profiles 502₁ . . . 502₃. FIGS. 11-13 shows the vectors corresponding to each of the three target areas (A, B, and C). More precisely, FIG. 11 shows the three vectors 1102 corresponding to the healthy skin test areas (A, B, and C) and the three vectors 1104 corresponding to the nodular melanoma test areas (A, B, and C). FIG. 12 shows the three vectors 1102 corresponding to the healthy skin test areas (A, B, and C) and the three vectors 1204 corresponding to the superficial spreading melanoma test areas (A, B, and C). FIG. 13 shows the three vectors 1102 corresponding to the healthy skin test areas (A, B, and C) and the three vectors 1304 corresponding to the melanoma metastasis test areas (A, B, and C). Each of the vectors 1102, 1104, 1204, and 1304, were generated by processor 110A as described above.

It is evident from FIGS. 11-13 that the vectors 1102 for normal healthy skin are separated from the cancerous tissue vectors 1104, 1204, and 1304. As one of ordinary skill will appreciate, a distance or separation between any two vectors can be easily calculated. As one of ordinary skill in the art will also appreciate, an average vector from a group of vectors can also be easily calculated. Thus, one may calculate an average vector v_{avg-1102} for the normal healthy skin sites and average vectors v_{avg-1104}, v_{avg-1204}, and v_{avg-1304}, for the cancerous sites. The differences between those averages vectors d₁₁₀₂₋₁₁₀₄, d₁₁₀₂₋₁₂₀₄, and d₁₁₀₂₋₁₃₀₄ can then be calculated. As is evident from FIGS. 11-13, the differences between the average vectors, d₁₁₀₂₋₁₁₀₄, d₁₁₀₂₋₁₂₀₄, and d₁₁₀₂₋₁₃₀₄, are non-negligible which shows that the system 100A was able to successfully discriminate between healthy skin and cancerous tissue. Non-negligible in this context means that the vectors are separated by, at least, an amount that lies outside of the accuracy of the system 100A. The accuracy of system 100A, however, is dependent on the accuracy of the cancerous vectors and how they were generated. The poorer the quality of the sampling that lead to the creation of the cancerous vector, the larger the margin of error there will be. If a difference between a vector corresponding to a target area 300 and a vector corresponding to a cancerous vector falls within the margin of error for the cancerous vector, then system 100A will return a match, and may also display on the notification device 112 the difference value along with the margin of error for the corresponding cancerous vector so that the provider may be aware of the possibility of a false positive result. In that case, memory 110B stores the margin of error for each cancerous vector. Having described the function of the optical filters 106₁ . . . 106₃ and the ability of the system 100A to discriminate between healthy tissue and cancerous, we will now return to FIG. 4 and the operation of the system 100A in a clinical setting.

Returning to FIG. 4, the filtered reflected light from filters 106₁ . . . 106₃ is directed onto the one or more optical sensors 108₁ (as the case may be). Sensors 108₁ generate voltages in proportion to the amount of filtered reflected light 202 that is received. Those voltages are a form of analog signal, which are converted into a digital signal which is analyzed by processor 110A. To recap, a suspected growth on a patient (target area 300) is illuminated by light 200 resulting in reflected light 202 which is filtered by optical filters 106₁ . . . 106₃ and recorded by one or more optical sensors 108. The data from the one or more optical sensors 108 is provided to controller 110, where processor 110A converts the data into vector components {v₁₀₆₋₁, v₁₀₆₋₂, v₁₀₆₋₃} for each of the optical filters. From the vector components, a three-dimensional vector v_{target-area} is easily computed. In the case where multiple locations of the suspected growth are scanned, like in FIGS. 7-13, multiple vectors are generated respectively corresponding to each scan site.

The computed vector(s) is then compared to a library of cancerous vectors stored in memory 110B that were generated by scanning a plurality of known cancerous tissues and analyzing them using the same optical filters 106₁ . . . 106₃ used in system 100A and the methods described above (S412). These vectors are used in S414 to determine whether the target area 300 includes cancerous tissue or normal healthy tissue. More specifically, a difference between the computed vector v_{target-area} and each of the cancerous vectors stored in memory 110B is calculated. For a given cancer type, if the difference between v_{target-area} and v_{cancer} (that is the separation between the two vectors) is non-negligible then the processor 110A determines that the target area 300 does not match that type of cancer. If, however, the difference between v_{target-area} and v_{cancer} is negligible, at least within the accuracy of system 100A, then processor 110A determines that target area 300 includes tissue of that cancer type. The computed vector v_{target-area} may also be compared to a vector for normal healthy skin. Like above, if the difference between those vectors is negligible, at least within the accuracy of the system 100A, then the processor determines that the target area 300 is normal healthy skin. In the event that system 100A is unable to produce a negligible result (meaning that no match was found) then the processor 110A determines that tissue is neither cancerous nor healthy skin and may be a precancerous growth.

Another analytical methodology enabling discrimination of cancerous tissue from non-cancerous tissue is described in the non-provisional patent application Ser. No. 16/571,461 titled “Infrared CIE Methodology for Chemical Group Classification”, which is incorporated by reference herein in its entirety. Briefly, this analytical methodology utilizes the same type of overlapping bandpass filters as described in PCT/US19/17166, however the infrared CIE methodology does not generate vectors. Because the approach for data collection, i.e., using three overlapping infrared optical filters, replicates how the human eye perceives color (patent application #16/571,461 section 0007) the output from the three channels corresponding to the three different infrared optical filters can be evaluated in the same manner as human color vision. In 1931 the Commission Internationale de l'Eclairage (CIE) developed the CIE color matching chart to quantify the response of the human eye to the full range of visible colors (1). The CIE chart describes the response of the human eye to all visible colors based on the interaction of the three photopigments (equivalent to optical filters) contained in the retina with visible light. The CIE chart consists of an operational area (color space) defined by the three photopigments contained within the retina. The response of the human eye to different colors, generated from the interaction of the three different retinal photopigments with different colors, are contained within this operational area.

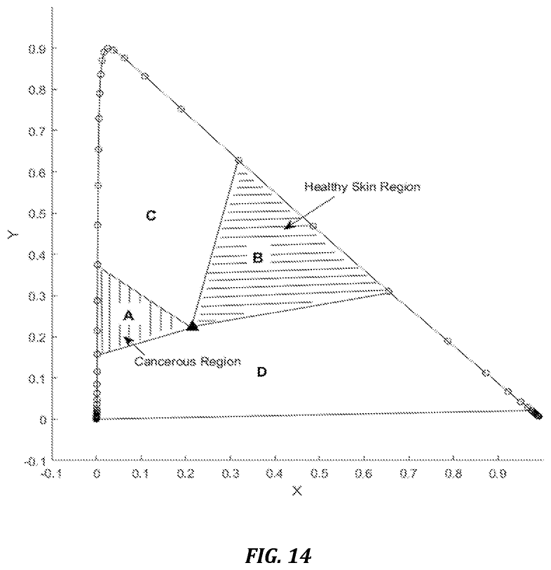

The CIE-Infrared methodology (CIE-IR) utilizes the output from the three different channels, corresponding to the three different infrared bandpass filters, in the same manner as the CIE chart utilizes the output from the three different photopigments in the eye. The CIE-IR methodology first defines an operational space for the set of IR filters used based on the three different infrared optical filters along with a weighting matrix. Note that this weighting matrix may also be equivalent to a “1/1” weighting. In this case, the operational space is solely defined by the optical filter spectral profiles. Once this operational space, termed the CIE-IR chart, is defined, unique regions are assigned to the CIE-IR chart where such regions are based on the response of the system to varying materials. The system response for materials of a given class are found within a given region, while materials of a different class are classified in a separate, defined region of the CIE-IR chart. These responses are based on the total power received for each filter as described in Ser. No. 16/571,461. The response for an unknown sample is determined, and plotted on the CIE-IR chart. This response will then intrinsically fall within a previously defined region of the CIE-IR chart—and thus the sample is identified as belonging to the class identified within that region. For the case of skin cancer detection, this would operate in practice as demonstrated in FIG. 14, which represents an illustrative example of skin cancer detection using the CIE-IR classification method. Here a region defined as indicating the presence of healthy skin has been defined (horizontal hashing), as well as a region indicating the presence of cancerous tissue (vertical hashing). If a response for an unknown sample resides at the point labelled “A” in FIG. 14, it would be identified as being cancerous tissue. Conversely, if a response for an unknown sample resides at the point labelled “B” in FIG. 14, it would be identified as being non-cancerous tissue. Finally, if a response for an unknown sample resides at the points labelled “C” or “D” in FIG. 14, it would not be able to be identified as either cancerous or non-cancerous within the acceptable statistical limits of this given CIE-IR chart, and thus would be classified as ‘inconconclusive.’ It should be readily apparent based on this description as well as the description in Ser. No. 16/571,461 that the CIE-IR chart and regions shown in FIG. 14 are for illustrative purposes only and other CIE-IR charts with varying regions (and varying n number of regions, so long as n>2) may also be used for identification of cancerous vs non-cancerous tissue, as well as for the discrimination between varying forms of cancer if such regions are defined on the CIE-IR chart.

Regardless of the determination in S414, the result is provided to notification device 112 which is then provided to the user (e.g., if notification device 112 is a screen, the result is displayed on the screen). Controller 110 may also, in one embodiment, provide the result of the determination to a connected device through I/O 122.

While various example embodiments of the invention have been described above, it should be understood that they have been presented by way of example, and not limitation. It is apparent to persons skilled in the relevant art(s) that various changes in form and detail can be made therein. Thus, the disclosure should not be limited by any of the above described example embodiments, but should be defined only in accordance with the following claims and their equivalents.

In addition, it should be understood that the figures are presented for example purposes only. The architecture of the example embodiments presented herein is sufficiently flexible and configurable, such that it may be utilized and navigated in ways other than that shown in the accompanying figures.

Further, the purpose of the Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the example embodiments presented herein in any way. It is also to be understood that the procedures recited in the claims need not be performed in the order presented.

What is claimed is:

1. A method of detecting cancerous tissue, comprising:
illuminating a target area comprising suspected tissue with infrared light;
receiving reflected light from the target area;
filtering the reflected light from the target through a plurality of mid-wavelength infrared optical filters with overlapping pass-bands;
directing the filtered reflected light from each of the plurality of optical infrared filters onto at least one optical sensor;
providing data generated by the at least one optical sensor and corresponding to the filtered reflected light from each of the plurality of optical infrared filters to a controller;
calculating, at the controller, a vector corresponding to the target area from the data;
comparing, at the controller, the vector corresponding to the target area with a plurality of vectors respectively corresponding to different types of cancerous tissue; and
determining, at the controller, based on a result of the comparison whether the target area includes at least one type of cancerous tissue.

2. The method of claim 1, wherein the infrared light is medium wavelength infrared light.

3. The method of claim 1, wherein the at least one optical sensor is: a PbSe image sensor, a Si microbolometer, a VOₓ microbolometer an infrared focal plane array, a HgCdTe detector, a deuterated triglycine sulfate detector, a InSb infrared photodiode optical sensor, or a InAs, infrared photodiode optical sensor.

4. The method of claim 1, wherein, in the comparing step, a plurality of differences between the vector corresponding to the target area and the plurality of vectors respectively corresponding to different types of cancerous tissues are calculated.

5. The method of claim 4, wherein for any of the plurality of differences that are negligible, the controller determines, in the determining step, that the target area includes a corresponding type of cancerous tissue.

6. An apparatus for detecting cancerous tissue, comprising:
an infrared light source configured to direct infrared light onto a target area comprising suspected tissue;
a plurality of mid-wavelength infrared optical filters configured to receive reflected light from the target area, wherein the plurality of infrared optical filters have overlapping pass-bands;
at least optical sensor configured to receive filtered reflected light from each of the plurality of optical infrared filters; and
a controller configured to:
receive data generated by the at least one optical sensor and corresponding to the filtered reflected light from each of the plurality of optical infrared filters;
calculate a vector corresponding to the target area from the data;
compare the vector corresponding to the target area with a plurality of vectors respectively corresponding to different types of cancerous tissue; and
determine based on a result of the comparison whether the target area includes at least one type of cancerous tissue.

7. The apparatus of claim 6, wherein the infrared light source emits medium wavelength infrared light.

8. The apparatus of claim 6, wherein the at least one optical sensor is: a Pb Se image sensor, a Si microbolometer, a VOₓ microbolometer an infrared focal plane array, a HgCdTe detector, a deuterated triglycine sulfate detector, a InSb infrared photodiode optical sensor, or a InAs, infrared photodiode optical sensor.

9. The apparatus of claim 6, wherein the controller is configured to compare the vector corresponding to the target area with the plurality of vectors respectively corresponding to different types of cancerous tissues by calculating a plurality of differences between the vector corresponding to the target area and the plurality of vectors respectively corresponding to different types of cancerous tissues.

10. The apparatus of claim 9, wherein the controller is configured such that for any of the plurality of differences that are negligible, the controller determines the target area includes a corresponding type of cancerous tissue.
